(19) United States
(12) Patent Application Publication
(10) Pub. No.: US 2023/0029576 A1
(43) Pub. Date: Feb. 2, 2023

(54) MEANS TO IMPROVE USABILITY OF A WOUND INSERT FOR APPLICATION TO DEEP WOUNDS

(71) Applicant: KCI Licensing, Inc., San Antonio, TX (US)

(72) Inventors: Christopher Brian LOCKE, Bournemouth (GB); Timothy Mark ROBINSON, Shillingstone (GB)

(21) Appl. No.: 17/790,944

(22) PCT Filed: Jun. 25, 2020
(86) PCT No.: PCT/IB2020/056043

§ 371 Date: Jul. 5, 2022

Related U.S. Application Data
(60) Provisional application No. 62/957,919, filed on Jan. 7, 2020.

(51) Int. Cl. A61F 13/36; A61L 15/22; A61L 15/64

(52) U.S. Cl. CPC A61F 13/36; A61L 15/225; A61L 15/64

(57) ABSTRACT

The present disclosure relates generally to wound inserts that may include an outer layer and an inner core of biopolymers that may be used in the therapy of tunneling wounds and for facilitating wound healing. Kits for use in practicing the methods are also provided.

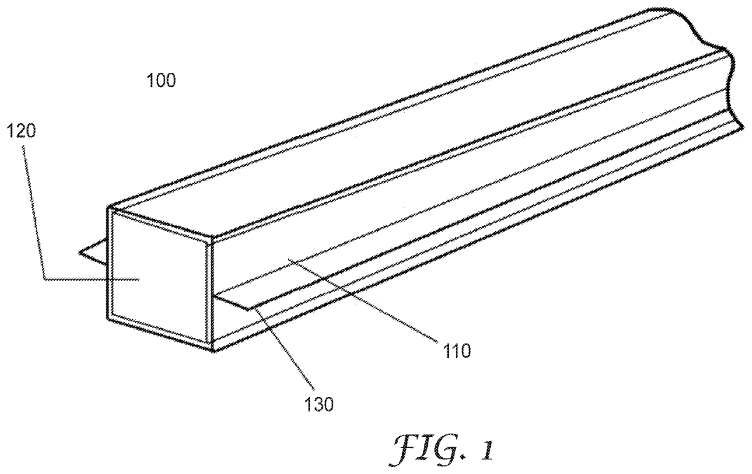

### TECHNICAL FIELD

The present technology relates generally to wound inserts and methods for facilitating wound healing. Kits for use in practicing the methods are also provided.

### BACKGROUND

The following description of the background of the present technology is provided simply as an aid in understanding the present technology and is not admitted to describe or constitute prior art to the present technology.

A wide variety of materials and devices, generally characterized as “dressings,” are generally known in the art for use in treating an injury or other disruption of tissue. Such wounds may be the result of trauma, surgery, or disease, and may affect skin or other tissues. In general, dressings may control bleeding, absorb wound exudate, ease pain, assist in debriding the wound, protect wound tissue from infection, or otherwise promote healing and protect the wound from further damage.

Healing lengthy tunneling wounds is very challenging since such wounds do not respond to mainstream wound care treatment. Wounds that do not respond to conventional treatment cannot only lead to a chronic wound, but also patient mortality. Application of conventional wound dressings to such wounds is limited due to loss of structural integrity presents additional challenges to treatment of tunneling wounds.

### SUMMARY

In an aspect, a wound insert is provided that includes an outer layer and an inner layer. The outer layer includes one or more bioabsorbable polymers, wherein the outer layer exhibits a modulus of elasticity of about 0.5 MPa to about 5.0 MPa, and the inner core includes a biopolymer, wherein the inner core has a lower modulus of elasticity than the outer layer.

In an aspect, methods for treating a wound in a subject in need thereof are provided, wherein the method includes administering a wound insert of any embodiment disclosed herein to the wound.

In a further related aspect, a method of manufacturing a wound insert of any embodiment of the present technology is provided, where the method includes: fabricating an inner core of the wound insert from a slurry that includes one or more biopolymers, where in the inner core is in the form of a rope or rod; drying the inner core; fabricating the outer layer that includes a reabsorbable polymer, wherein the outer layer is shaped around the inner core; wherein: the wound insert is an a shape comprising a cylinder, a sphere, a cube, a cuboid, a hexagonal prism, a cone, a square-based pyramid, triangular-based pyramid, or a triangular prism; the outer layer has a modulus of elasticity from about 0.5 MPa to about 5.0 MPa; and the inner core exhibits a lower modulus of elasticity than the outer layer.

In a further related aspect, the present disclosure provides kits that include a wound insert of any embodiment disclosed herein and instructions for use.

### BRIEF DESCRIPTION OF THE DRAWINGS

The FIGURE provides a non-limiting representative illustration of an embodiment of a wound insert of the present technology, illustrating the outer layer and the inner core of such a wound insert.

### DETAILED DESCRIPTION

It is to be appreciated that certain aspects, modes, embodiments, variations, and features of the present methods are described below in various levels of detail in order to provide a substantial understanding of the present technology.

### Definitions

The definitions of certain terms as used in this specification are provided below. Unless defined otherwise, all technical and scientific terms used herein generally have the same meaning as commonly understood by one of ordinary skill in the art to which this present technology belongs.

The following terms are used throughout as defined below.

As used herein and in the appended claims, singular articles such as “a”, “an”, and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as discloses “10 wt. %.”

As used herein, the term “modulus of elasticity” (also known as “Young's modulus”) refers to the measure of an object or a substance's resistance to being deformed elastically (i.e., non-permanently) upon the application of a stress. The modulus of elasticity of an object is defined by the following formula:

$$E = \frac{tensile stress ( σ )}{tensile strain ( ε )}$$

wherein “stress” is the force causing the deformation divided by the area to which the force is applied, and wherein “strain” is the ratio of the change in some parameter caused by the deformation to the original value of the parameter.

As used herein, the term “mammalian recombinant collagen” refers to collagen manufactured by culturing a non-human organism or mammalian or non-mammalian cells to express at least one exogenous gene encoding a collagen in the culturing system. The term “human recombinant collagen” refers to collagen manufactured by culturing a non-human organism or mammalian or non-mammalian cells to express at least one human gene encoding a collagen. The human recombinant collagen may be selected from the group consisting of collagen type I, type II, type III, type IV, type V, type VI, type VII, type VIII, type IX, type X, type XI, type XII, type XIII, type XIV, type XV, type XVI, type XVII, type XVIII, type XIX, type XX, type XXI, type XXII, type XXIII, type XXIV, type XXV, type XXVI, type XXVII, and mixtures of any two or more thereof. The human recombinant collagen can be collagen of one type free of any other type, or can be a mixture of collagen types. Suitably, the human recombinant collagen includes collagens selected from collagen type I, collagen type II, or mixtures thereof. The term “bovine recombinant collagen” refers to collagen manufactured by culturing a non-human organism or mammalian or non-mammalian cells to express at least one, bovine gene encoding a collagen. The bovine recombinant collagen may be selected from collagen type I, type II, type III, type IV, or mixtures of any two or more thereof. The bovine recombinant collagen can be collagen of one type free of any other type, or can be a mixture of collagen types. Suitably, the bovine recombinant collagen includes collagens selected from collagen type I, collagen type III, and mixtures thereof.

As used herein, the “administration” of a wound insert to a subject includes any route of introducing or delivering to a subject a wound insert to perform its intended function. Administration can be carried out by any suitable route, including, but not limited to, topical administration. Administration includes self-administration and the administration by another.

As used herein, the term “effective amount” refers to a quantity sufficient to achieve a desired therapeutic and/or prophylactic effect, e.g., an amount which results in the decrease in a wound described herein or one or more signs or symptoms associated with a wound described herein. In the context of therapeutic and/or prophylactic applications, the amount of an insert administered to the subject will vary depending on the composition, the degree, type, and severity of the wound and on the characteristics of the individual. The inserts can also be administered in combination with one or more additional therapeutic compounds. In the methods described herein, the therapeutic inserts may be administered to a subject having one or more wounds.

As used herein, the terms “individual”, “patient”, or “subject” can refer to an individual organism, a vertebrate, a mammal, or a human. In some embodiments, the individual, patient, or subject is a human.

“Treating” or “treatment” as used herein covers the treatment of a wound described herein, in a subject, such as a human, and includes: (i) inhibiting a wound, i.e., arresting its development; (ii) relieving a wound, i.e., causing regression of the wound; (iii) slowing progression of the wound; and/or (iv) inhibiting, relieving, or slowing progression of one or more symptoms of the wound. In some embodiments, treatment means that the symptoms associated with the wound are alleviated, reduced, cured, or placed in a state of remission.

It is also to be appreciated that the various modes of treatment of wounds as described herein are intended to mean “substantial,” which includes total but also less than total treatment, and wherein some biologically or medically relevant result is achieved. The treatment may be a continuous prolonged treatment for a chronic wound or a single administration, or a few administrations for the treatment of an acute wound.

### The Present Technology

A very lengthy tunneling wound is very challenging to heal since such wounds do not respond to mainstream wound care treatment. The complex topographies of such wounds present challenges to conventional wound dressing materials, as the structural integrity of such dressings deteriorate upon application to a tunneling wound.

The present disclosure is directed to wound inserts that include an outer layer and an inner core of biopolymers. The wound inserts of the present technology advantageously may help prevent the collapse of the wound. The components of the wound insert may stimulate tissue growth and/or prevent bacterial infection. The components of the wound insert may reduce the inflammatory phase, may stimulate granulation tissue growth, and/or may reduce bacterial bioburden.

Thus, in an aspect, a wound insert is provided that includes an outer layer and an inner layer. The outer layer includes one or more bioabsorbable polymers, wherein the outer layer exhibits a modulus of elasticity of about 0.5 MPa to about 5.0 MPa, and the inner core includes a biopolymer, wherein the inner core has a lower modulus of elasticity than the outer layer. In any embodiment disclosed herein, the outer layer may exhibits a modulus of elasticity of about 0.5 MPa, about 0.6 MPa, about 0.7 MPa, about 0.8 MPa, about 0.9 MPa, about 1.0 MPa, about 1.3 MPa, about 1.4 MPa, about 1.5 MPa, about 1.6 MPa, about 1.7 MPa, about 1.8 MPa, about 1.9 MPa, about 2.0 MPa, about 2.1 MPa, about 2.2 MPa, about 2.3 MPa, about 2.4 MPa, about 2.5 MPa, about 2.6 MPa, about 2.7 MPa, about 2.8 MPa, about 2.9 MPa, about 3.0 MPa, about 3.1 MPa, about 3.2 MPa, about 3.3 MPa, about 3.4 MPa, about 3.5 MPa, about 3.6 MPa, about 3.7 MPa, about 3.8 MPa, about 3.9 MPa, about 4.0 MPa, about 4.1 MPa, about 4.2 MPa, about 4.3 MPa, about 4.4 MPa, about 4.5 MPa, about 4.6 MPa, about 4.7 MPa, about 4.8 MPa, about 4.9 MPa, about 5.0 MPa, or any range including and/or in between any two of these values. For example, in any embodiment disclosed herein, the outer layer may exhibit a modulus of elasticity of about 0.5 MPa to about 5.0 MPa, about 1.0 MPa to about 2.5 MPa, or about 1.2 MPa to about 2.3 MPa. As discussed above, the inner core exhibits a lower modulus of elasticity than the outer layer. In any embodiment disclosed herein, the inner core may exhibit a modulus of elasticity of about 0.0 MPa, about 0.1 MPa, about 0.2 MPa, about 0.3 MPa, about 0.5 MPa, or any range including and/or in between any two of these values. The FIGURE provides a non-limiting representative illustration of an embodiment of a wound insert of the present technology, illustrating the outer layer and the inner core of such a wound insert. The FIGURE provides an illustrative profile view illustration of a wound insert 100 having a cube shape that includes an outer layer 110, an inner core 120, and a weld/joint 130 formed from excess outer layer.

### The Outer Layer

In any embodiment disclosed herein, the outer layer may include an inner core facing side and a side facing away from the inner core (i.e., outer facing side). The outer-facing side of the outer layer may be configured to be in contact with a wound when in use. In any embodiment disclosed herein, it may be that the outer layer envelopes the inner core.

The thickness of the outer layer of any embodiment herein may be about 20 μM to about 300 μM. Thus, the thickness of the outer layer may be about 20 μM, about 22 μM, about 24 μM, about 26 μM, about 28 μM, about 30 μM, about 32 μM, about 34 μM, about 36 μM, about 38 μM, about 40 μM, about 42 μM, about 44 μM, about 46 μM, about 48 μM, about 50 μM, about 55 μM, about 60 μM, about 65 μM, about 70 μM, about 75 μM, about 80 μM, about 85 μM, about 90 μM, about 95 μM, about 100 μM, about 110 μM, about 120 μM, about 130 μM, about 140 μM, about 150 μM, about 160 μM, about 170 μM, about 180 μM, about 190 μM, about 200 μM, about 220 μM, about 240 μM, about 260 μM, about 280 μM, about 300 μM, or any range including and/or in between any two of these values.

The bioabsorbable polymer of the outer layer may include polylactic acid (PLA), gelatin, polyglycolide, polycaprolactone, or a combination of any two or more thereof. The outer layer may include about 0.01 weight percent (“wt. %”) to about 100 wt. % of the bioabsorbable polymer. Thus, the outer layer may include bioabsorbable polymer in an amount (based on the total weight of the outer layer) of about 0.01 wt. %, about 0.1 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 12 wt. %, about 14 wt. %, about 16 wt. %, about 18 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 95 wt. %, 100 wt. %, or any range including and/or in between any two of these values. In any embodiment herein, the outer layer may include PLA, gelatin, or a combination thereof. For example, in any embodiment disclosed herein, the outer layer may include PLA. Additionally or alternatively, in some embodiments, the outer layer may include gelatin.

In any embodiment disclosed herein, the outer layer may further include one or more of collagen, plasticizers, functional agents, or a combination of two or more thereof.

In any embodiment disclosed herein, the collagen of the outer layer may include a mammalian collagen, such as a bovine collagen, a human collagen, or a combination thereof. The collagen of any embodiment herein may be a Type I collagen, a Type II collagen, a Type III collagen, may be obtained from any natural source, may be chemically-modified collagen (e.g., an atelocollagen obtained by removing the immunogenic telopeptides from natural collagen), or may be a combination of any two or more thereof. For example, the collagen may include collagen obtained from bovine corium that has been rendered largely free of non-collagenous components, for example, including fat, non-collagenous proteins, polysaccharides, and other carbohydrates, such as by procedures described in U.S. Pat. Nos. 4,614,794 and 4,320,201, each of which is incorporated herein by reference. The bovine collagen may include one or both of bovine collagen type I and bovine collagen type III. In any embodiment disclosed herein, the outer layer may include a weight ratio of human collagen type I to human collagen type III of about 100:0, about 90:10, about 80:20, about 70:30, about 60:40, about 50:50, about 40:60, about 30:70, about 20:80, about 10:90, about 0:100, or any range including and/or in between any two of these values. The ratio by weight of human collagen type I to human collagen type III may be greater than about 50:50, or greater than about 70:30. The collagen of any embodiment herein may include a weight ratio of type I bovine collagen to type III bovine collagen of about 95:5, about 85:15, about 75:25, about 65:35, about 55:45, about 50:50, about 45:55, about 65:35, about 75:25, about 85:15, about 95:5, or any range including and/or in between any two of these values. The ratio by weight of the type I bovine collagen to type III bovine collagen may be about 85:15.

In any embodiment disclosed herein, the outer layer includes about 0.001 wt. % to about 10 wt. % of collagen based on the weight of the outer layer. Thus, the total amount of collagen in the outer layer may be about 0.001 wt. %, about 0.002 wt. %, about 0.003 wt. %, about 0.004 wt. %, about 0.005 wt. %, about 0.006 wt. %, about 0.007 wt. %, about 0.008 wt. %, about 0.009 wt. %, about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, or any range including and/or in between any two of these values.

The collagen included in the outer layer of any embodiment herein may have a weight-average molecular weight of about 5,000, about 6,000, about 7,000, about 8,000, about 9,000, about 10,000, about 12,000, about 14,000, about 16,000, about 18,000, about 20,000, about 22,000, about 24,000, about 26,000, about 28,000, about 30,000, about 32,000, about 34,000, about 36,000, about 38,000, about 40,000, about 45,000, about 50,000, about 55,000, about 60,000, about 65,000, about 70,000, about 75,000, about 80,000, about 85,000, about 90,000, about 95,000, about 100,000, or any range including and/or in between any two of these values.

### The Inner Core

In any embodiment disclosed herein, the inner core may include a wound-facing side.

The biopolymer of the inner core may be a collagen, an oxidized cellulose, an oxidized regenerated cellulose (ORC), a polysaccharide, chitosan, hyaluronic acid, or a combination of any two or more thereof. The inner core may include about 0.01 wt. %, about 0.1 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. % to about 100 wt. % of the biopolymer; thus, the inner core may include the biopolymer at an amount (based on the weight of the inner core) of about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 95 wt. %, about 99 wt. %, about 100 wt. %, or any range including and/or in between any two of these values based on weight of the inner core. For example, in any embodiment disclosed herein, the inner core may include from about 1 wt. % to about 80 wt. %, about 5 wt. % to about 65 wt. %, about 10 wt. % to about 50 wt. %, or any range including and/or in between any two of the preceding values based on the weight of the inner core.

In any embodiment disclosed herein, the inner core may include collagen. Additional or alternatively, in some embodiments, the inner core may include collagen and ORC.

In any embodiment disclosed herein, the collagen of the inner core may include a mammalian collagen, such as a bovine collagen, a human collagen, or a combination thereof. The collagen of any embodiment herein may be a Type I collagen, a Type II collagen, a Type III collagen, may be obtained from any natural source, may be chemically-modified collagen (e.g., an atelocollagen obtained by removing the immunogenic telopeptides from natural collagen), or may be a combination of any two or more thereof. For example, the collagen may include collagen obtained from bovine corium that has been rendered largely free of non-collagenous components, for example, including fat, non-collagenous proteins, polysaccharides, and other carbohydrates, such as by procedures described in U.S. Pat. Nos. 4,614,794 and 4,320,201, each of which is incorporated herein by reference. The bovine collagen may include one or both of bovine collagen type I and bovine collagen type III. In any embodiment disclosed herein, the inner core may include a weight ratio of human collagen type I to human collagen type III of about 100:0, about 90:10, about 80:20, about 70:30, about 60:40, about 50:50, about 40:60, about 30:70, about 20:80, about 10:90, about 0:100, or any range including and/or in between any two of these values. The ratio by weight of human collagen type I to human collagen type III may be greater than about 50:50, or greater than about 70:30. The collagen of any embodiment herein may include a weight ratio of type I bovine collagen to type III bovine collagen of about 95:5, about 85:15, about 75:25, about 65:35, about 55:45, about 50:50, about 45:55, about 65:35, about 75:25, about 85:15, about 95:5, or any range including and/or in between any two of these values. The ratio by weight of the type I bovine collagen to type III bovine collagen may be about 85:15.

The amount of collagen included in the inner core of any embodiment herein may be about 30 wt. % to 100 wt. %. Thus, the amount of collagen may be about 30 wt. %, about 32 wt. %, about 34 wt. %, about 36 wt. %, about 38 wt. %, about 40 wt. %, about 42 wt. %, about 44 wt. %, about 46 wt. %, about 48 wt. %, about 50 wt. %, about 52 wt. %, about 54 wt. %, about 56 wt. %, about 58 wt. %, about 60 wt. %, about 62 wt. %, about 64 wt. %, about 66 wt. %, about 68 wt. %, about 70 wt. %, about 72 wt. %, about 74 wt. %, about 76 wt. %, about 78 wt. %, about 80 wt. %, about 82 wt. %, about 84 wt. %, about 86 wt. %, about 88 wt. %, about 90 wt. %, about 92 wt. %, about 94 wt. %, about 95 wt. %, about 96 wt. %, about 97 wt. %, about 98 wt. %, about 99 wt. %, or any range including and/or in between any two of these values.

The collagen included in the inner core of any embodiment herein may have a weight-average molecular weight of about 5,000, about 6,000, about 7,000, about 8,000, about 9,000, about 10,000, about 12,000, about 14,000, about 16,000, about 18,000, about 20,000, about 22,000, about 24,000, about 26,000, about 28,000, about 30,000, about 32,000, about 34,000, about 36,000, about 38,000, about 40,000, about 45,000, about 50,000, about 55,000, about 60,000, about 65,000, about 70,000, about 75,000, about 80,000, about 85,000, about 90,000, about 95,000, about 100,000, or any range including and/or in between any two of these values.

As discussed above, the inner core may include ORC. ORC may be produced by the oxidation of cellulose, for example with dinitrogen tetroxide and/or as described in U.S. Pat. No. 3,122,479 (incorporated herein by reference). Not intending to be bound by theory, it is believed that this process may convert primary alcohol groups on the saccharide residues of the cellulose to carboxylic acid groups, for example, forming uronic acid residues within the cellulose chain. The oxidation may not proceed with complete selectivity, and as a result hydroxyl groups on carbons 2 and 3 of the saccharide residue may be converted to the keto form. These ketone units may introduce an alkali labile link, which at pH 7 or higher initiates the decomposition of the polymer via formation of a lactone and sugar ring cleavage. As a result, oxidized regenerated cellulose is biodegradable and bioresorbable under physiological conditions. ORC is available with a variety of degrees of oxidation and hence rates of degradation. The ORC may include particles, fibers, or both; in any embodiment disclosed herein, the ORC may be in the form of particles, such as fiber particles or powder particles. In embodiments that include ORC fibers, the ORC fibers may have a volume fraction such that at least 80% of the fibers have lengths in the range from about 5 μm to about 1000 μm, or from about 250 μm to about 450 μm.

The inner core of any embodiment herein may include about 30 wt. % to about 70 wt. % ORC with a weight-average molecular weight of about 50,000 to about 1,000,000. Thus, the ORC of any embodiment disclosed herein may be included in an amount of about 30 wt. %, about 32 wt. %, about 34 wt. %, about 36 wt. %, about 38 wt. %, about 40 wt. %, about 42 wt. %, about 44 wt. %, about 46 wt. %, about 48 wt. %, about 50 wt. %, about 52 wt. %, about 54 wt. %, about 56 wt. %, about 58 wt. %, about 60 wt. %, about 62 wt. %, about 64 wt. %, about 66 wt. %, about 68 wt. %, about 70 wt. %, or any range including and/or in between any two of these values. The ORC may have a weight-average molecular weight of about 50,000, about 100,000, about 150,000, about 200,000, about 250,000, about 300,000, about 350,000, about 400,000, about 450,000, about 500,000, about 550,000, about 600,000, about 650,000, about 700,000, about 750,000, about 800,000, about 850,000, about 900,000, about 950,000, about 1,000,000, or any range including and/or in between any two of these values.

In any embodiment herein including collagen along with ORC in the inner core, a weight ratio of collagen to ORC may be about 60:40 to about 40:60 in the inner core. The weight ratio of collagen to ORC may be about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, or any range including and/or in between any two of these values.

In any embodiment disclosed herein, the inner core may further include one or more of bioabsorbable polymers, cross-linking agents, plasticizers, functional agents, or combinations of any two or more thereof.

In any embodiment disclosed herein, the inner core may include about 1 wt. % to about 20 wt. % of bioresorbable polymer based on the weight of the inner core. Exemplary bioresorbable polymer may include, but are not limited to, polylactic acid (PLA), gelatin, polyglycolide, polycaprolactone, or a combination of any two or more thereof. Thus, the total amount of the bioresorbable polymer in the inner core may be about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, or any range including and/or in between any two of these values based on the weight of the inner core.

In any embodiment disclosed herein, the inner core may include about 0.01 wt. % to about 10 wt. % of cross-linking agents based on the weight of the inner core. Exemplary cross-linking agents may include, but are not limited to, epichlorhydrin, carbodiimide, hexamethylene diisocyanate (HMDI) orglutaraldehyde, or a combination of any two or more thereof. Thus, the total amount of the cross-linking agents may include in the inner core may be about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, or any range including and/or in between any two of these values based on the weight of the inner core.

In any embodiment herein, the inner core may further include powders, porous matrices, or combinations thereof. Such powders in any embodiment herein, for example, may include freeze-dried collagen/ORC in powder form. Such porous matrix materials may include a freeze-dried collagen/ORC matrix.

In any embodiment disclosed herein, the inner core may be in the form of a rope or rod. For example, in any embodiment disclosed herein, the rope and/or rod form of the inner core conforms to the overall shape of the wound insert.

Features of the Outer Layer and/or Inner Core

In any embodiment disclosed herein, the outer layer and/or inner core may each independently include about 0.001 wt. % to about 5 wt. % of functional agents (by weight of the outer layer and/or inner core, respectively). Thus, the total amount of functional agents in the outer layer and/or the inner core may independently for each be about 0.001 wt. %, about 0.002 wt. %, about 0.003 wt. %, about 0.004 wt. %, about 0.005 wt. %, about 0.006 wt. %, about 0.007 wt. %, about 0.008 wt. %, about 0.009 wt. %, about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, or any range including and/or in between any two of these values.

In any embodiment disclosed herein, the one or more functional agents of the outer layer and/or inner core may include antimicrobial agents, growth factors, peptides, antioxidants, or combinations of any two or more thereof. Exemplary antimicrobial agent(s) may include a penicillin, a streptomycin, ionic silver (or a source for ionic silver), chlorhexidine, a poly(hexamethylene biguanide) (PHMB), iodine, or a combination of any two or more thereof. Suitable sources of ionic silver may include ionic silver in a variety of forms including as pharmaceutically acceptable salts, where representative examples include but are not limited to silver oxide, silver chromate, silver allantoinate, silver borate, silver glycerolate, silver nitrate, silver acetate, silver chloride, silver sulfate, silver lactate, silver bromide, silver iodide, silver carbonate, silver citrate, silver laurate, silver deoxycholate, silver salicylate, silver p-aminobenzoate, silver p-aminosalicylate, a silver oxysalt, or a combination of any two or more thereof.

In any embodiment disclosed herein, the outer layer and/or the inner core may each independently include one or more growth factors. Exemplary growth factors in the outer layer and/or inner core may include, but are not limited to, one or more of fibroblast growth factors (FGFs), insulin-like growth factor (IGF), keratinocyte growth factor (KGF), vascular endothelial growth factor (VEGF), transforming growth factor β (TGFβ), epidermal growth factor (EGF), hepatocyte growth factor (HGF), or platelet-derived growth factor (PDGF). Additionally or alternatively, in some embodiments, the fibroblast growth factors comprise one or more of fibroblast growth factor 1 (FGF1), fibroblast growth factor 2 (FGF2), fibroblast growth factor 3 (FGF3), fibroblast growth factor 4 (FGF4), fibroblast growth factor 5 (FGF5), fibroblast growth factor 6 (FGF6), fibroblast growth factor 7/keratinocyte growth factor (FGF7/KGF), fibroblast growth factor 8 (FGF8), fibroblast growth factor 9 (FGF9), fibroblast growth factor 10/keratinocyte growth factor 2 (FGF10/KGF2), fibroblast growth factor 11 (FGF11), fibroblast growth factor 12 (FGF12), fibroblast growth factor 13 (FGF13), fibroblast growth factor 14 (FGF14), fibroblast growth factor 15 (FGF15), fibroblast growth factor 16 (FGF16), fibroblast growth factor 17 (FGF17), fibroblast growth factor 18 (FGF18), fibroblast growth factor 19 (FGF19), fibroblast growth factor 20 (FGF20), fibroblast growth factor 21 (FGF21), fibroblast growth factor 22 (FGF22), fibroblast growth factor 23 (FGF23), or any combination thereof.

In any embodiment disclosed herein, the outer layer and/or the inner core may each independently include one or more peptides; for example, in any embodiment disclosed herein, the one or more peptides may include defensins, histatins, cathelicidin LL-37, or any combination of two or more thereof.

In any embodiment disclosed herein, the outer layer and/or the inner core may each independently include one or more antioxidants. Exemplary antioxidants include, but are not limited to, anthocyanins, astaxanthin, bilirubin, canthaxanthin, capsaicin, curcumin, coenzyme Q10, eugenol, flavanol, flavonolignans, flavanone, flavone, flavonol, iodide, isoflavone phytoestrogen, lutein, lycopene, manganese, melatonin, N-acetylcysteine, oxalic acid, phenolic acid, phytic acid, R-α-lipoic acid, stilbenoid, tocopherol, tocotrienol, vitamin A, vitamin C, vitamin E, xanthones, zeaxanthin, α-carotene, β-carotene, as well as a combination of any two or more thereof.

In any embodiment disclosed herein, the outer layer and/or the inner core may include about 1 wt. % to about 20 wt. % of plasticizers. Thus, the total amount of plasticizers in the outer layer and/or the inner layer may each independently be about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, or any range including and/or in between any two of these values. Exemplary plasticizers in any embodiment disclosed herein may include, but are not limited to, glycerin, polyethylene oxide, or a combination thereof.

The outer layer and inner core may contain varying concentration of any of the above referenced materials (e.g., functional agents, plasticizers, and/or additional materials as described herein in any embodiment). For example, in any embodiment herein, both the outer layer and inner core may include one or more functional agents as described herein, where the outer layer may include a higher concentration of functional agents than the inner core. In any embodiment herein, both the outer layer and inner core may include one or more functional agents as described herein, where the outer layer may include a lower concentration of functional agents than the inner core.

### The Wound Insert

As provided previously, the present technology provides a wound insert that includes an outer layer and an inner core. The outer layer may include a wound-facing side and an inner-core facing side. In any embodiment disclosed herein, an exterior surface of the inner core may be adjoined with the inner core-facing side of the outer layer.

In any embodiment disclosed herein, the wound insert of the present technology may be in the shape of a cylinder, a sphere, a cube, a cuboid, a hexagonal prism, a cone, a square-based pyramid, triangular-based pyramid, or a triangular prism. For example, the wound insert of the present technology may be in the shape of a cylinder, a cylinder with at least one rounded edge, cuboid, or a cuboid with at least one rounded edge. In any embodiment herein, the wound insert may be in the shape of a cube, wherein the cube wound insert further comprises a excess outer layer material forming a weld or joint portions (see FIGURE). In any embodiment herein, the wound insert may be in the shape of a cuboid with one or more rounded edges, wherein the cuboid wound insert further comprises a cubed compartment on each end of the wound insert.

In any embodiment disclosed herein, the length of the wound insert of the present technology compared to the width may be from about 15:1 to about 15:6. For example, in any embodiment herein, the length to width ratio may be about 15:1, about 15:2, about 15:3, about 15:4, about 15:5, about 15:6, or any range including and/or in between any two of the preceding values. In any embodiment disclosed herein, the width to height ratio of the wound insert according to the present technology may be about 3:2 to 2:3. For example, in any embodiment herein, the width to height ratio may be about 3:2, about 3:1, about 2:1, about 1:1, about 1:2, about 1:3, about 2:3, or any range including and/or in between any two of the preceding values.

In any embodiment disclosed herein, the length of the wound insert of the present technology may be about 1 cm to about 10 cm. Thus, the length of the wound insert may be about 1 cm, about 1.1 cm, about 1.2 cm, about 1.3 cm, about 1.4 cm, about 1.5 cm, about 1.6 cm, about 1.7 cm, about 1.8 cm, about 1.9 cm, about 2.0 cm, about 2.2 cm, about 2.4 cm, about 2.6 cm, about 2.8 cm, about 3.0 cm, about 3.2 cm, about 3.4 cm, about 3.6 cm, about 3.8 cm, about 4.0 cm, about 4.2 cm, about 4.4 cm, about 4.6 cm, about 4.8 cm, about 5.0 cm, about 5.5 cm, about 6.0 cm, about 6.5 cm, about 7.0 cm, about 7.5 cm, about 8.0 cm, about 8.5 cm, about 9.0 cm, about 9.5 cm, about 10.0 cm, or any range including and/or in between any two of these values.

In any embodiment disclosed herein, the width and/or height of the wound insert of the present technology may be about 0.1 cm to about 3 cm. Thus, the width and/or height of the wound insert may be about 0.1 cm, about 0.2 cm, about 0.3 cm, about 0.4 cm, about 0.5 cm, about 0.6 cm, about 0.7 cm, about 0.8 cm, about 0.9 cm, about 1.0 cm, about 1.1 cm, about 1.2 cm, about 1.3 cm, about 1.4 cm, about 1.5 cm, about 1.6 cm, about 1.7 cm, about 1.8 cm, about 1.9 cm, about 2.0 cm, about 2.1 cm, about 2.2 cm, about 2.3 cm, about 2.4 cm, about 2.5 cm, about 2.6 cm, about 2.7 cm, about 2.8 cm, about 2.9 cm, about 3.0 cm, or any range including and/or in between any two of these values.

In any embodiment disclosed herein, the wound insert of the present technology may include perforations to facilitate tissue integration. The perforations of the wound insert may be about 0.1 μm to about 100 μm in width and/or height. The perforations of the wound insert may be about 0.1 μm to about 100 μm deep. The perforations of the wound insert may be through the entire thickness of the outer layer. Additionally or alternatively, in some embodiments, the outer layer of the wound insert may be a mesh of the bioabsorbable polymer as disclosed herein in any embodiment.

In any embodiment disclosed herein, the wound insert of the present technology may include through-holes through the entire width, height, and/or length of the wound insert, such as for beneficial use of the wound insert in negative pressure therapy. The through-holes of the wound insert may be about 2 mm to about 8 mm in width and/or height. Thus, the through-holes of the wound insert may be about 2 mm, about 2.2 mm, about 2.4 mm, about 2.6 mm, about 2.8 mm, about 3 mm, about 3.2 mm, about 3.4 mm, about 3.6 mm, about 3.8 mm, about 4 mm, about 4.2 mm, about 4.4 mm, about 4.6 mm, about 4.8 mm, about 5 mm, about 5.5 mm, about 6 mm, about 6.5 mm, about 7 mm, about 7.5 mm, about 8 mm, or any range including and/or in between any two of these values.

In any embodiment disclosed herein, the wound insert of the present disclosure may be sterile and packaged in a microorganism-impermeable container.

### Negative-Pressure Therapy

The wound insert of any embodiment described herein may be employed in therapy in which a wound is treated with reduced pressure. Treatment of a wound with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and/or micro-deformation of tissue at a wound site. Together, these benefits may increase development of granulation tissue and reduce healing times.

Generally, the system may be configured to provide negative-pressure to a wound in accordance with this specification. In any embodiment herein, the system may generally include a negative-pressure supply, and may include or be configured to be coupled to a distribution component. In general, a distribution component may refer to any complementary or ancillary component configured to be fluidly coupled to a negative-pressure supply in a fluid path between a negative-pressure supply and a wound.

In any embodiment herein, the wound insert may be configured to distribute negative pressure. Additionally or alternatively, the fluid path(s) may be reversed or a secondary fluid path may be provided to facilitate movement of fluid across a wound. Additionally or alternatively, the fluid pathways of the through-holes may be interconnected to improve distribution or collection of fluids.

The fluid mechanics associated with using a negative-pressure source to reduce pressure in another component or location, such as within a sealed therapeutic environment, can be mathematically complex. However, the basic principles of fluid mechanics applicable to negative-pressure therapy are generally well-known to those skilled in the art. The process of reducing pressure may be described generally and illustratively herein as “delivering,” “distributing,” or “generating” negative pressure, for example.

In general, a fluid, such as wound fluid (for example, wound exudates and other fluids), flows toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. This orientation is generally presumed for purposes of describing various features and components herein. However, the fluid path may also be reversed in some applications (such as by substituting a positive-pressure source for a negative-pressure source) and this descriptive convention should not be construed as a limiting convention.

“Negative pressure” may generally refer to a pressure less than a local ambient pressure, such as the ambient pressure in a local environment external to a sealed therapeutic environment provided by the wound insert. In many cases, the local ambient pressure may also be the atmospheric pressure proximate to or about a wound. Alternatively or additionally, the pressure may be less than a hydrostatic pressure associated with the tissue at the wound. While the amount and nature of negative pressure applied to a wound may vary according to therapeutic requirements, the pressure is generally a low vacuum, also commonly referred to as a rough vacuum, between −5 mm Hg (−667 Pa) and −500 mm Hg (−66.7 kPa), gauge pressure. Common therapeutic ranges are between −50 mm Hg (−6.7 kPa) and −300 mm Hg (−39.9 kPa), gauge pressure.

Additionally or alternatively, in any embodiment herein, a negative-pressure supply may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components, such as sensors, processing units, alarm indicators, memory, databases, software, display devices, or user interfaces that further facilitate therapy. A negative-pressure source may be combined with a controller and other components into a therapy unit. A negative-pressure supply may also have one or more supply ports configured to facilitate coupling and de-coupling of the negative-pressure supply to one or more distribution components.

In any embodiment herein, components may be fluidly coupled to each other to provide a path for transferring fluids (i.e., liquid and/or gas) between the components. For example, components may be fluidly coupled through a fluid conductor, such as a tube. As used herein, the term “fluid conductor” may include a tube, pipe, hose, conduit, or other structure with one or more lumina or open passages adapted to convey a fluid between two ends thereof. Typically, a fluid conductor may be an elongated, cylindrical structure with some flexibility, but the geometry and rigidity may vary. Additionally or alternatively, in any embodiment herein, the negative-pressure source may be operatively coupled to the wound insert via an interface.

### Treatment Methods of the Present Technology

In an aspect, methods for treating a wound in a subject in need thereof are provided, wherein the method includes administering a wound insert of any embodiment disclosed herein to the wound. The wound may be a tunneling wound, a dermal wound, a diabetic wound, an acute wound, a chronic wound, or a combination of any two or more thereof. Exemplary chronic wounds include, but are not limited to, infectious wounds, venous ulcers, decubitus ulcers, or diabetic ulcers. In any embodiment herein, the wound is a tunneling wound. The method may include administering two or more wound inserts to the wound.

The wound insert may be administered directly to the wound. Any method known to those in the art for administering a wound insert to a tunneling wound, a dermal wound, a diabetic wound, an acute wound, or a chronic wound disclosed herein may be employed. Suitable methods include in vitro or in vivo methods. In vivo methods typically include the administration of one or more wound inserts to a subject in need thereof, suitably a human. When used in vivo for therapy, the one or more wound inserts described herein are administered to the subject in effective amounts (i.e., amounts that have desired therapeutic effect). The dose and dosage regimen will depend upon the state of the wound of the subject and the characteristics of the particular wound insert used.

The effective amount may be determined during pre-clinical trials and clinical trials by methods familiar to physicians and clinicians. An effective amount of one or more wound inserts useful in the methods may be administered to a subject in need thereof by any number of well-known methods for administering wound inserts.

In any embodiment disclosed herein, the wound inserts may be administered daily for 1 hour or more, for 2 hours or more, for 3 hours or more, for 4 hours or more, for 5 hours or more, for 6 hours or more, or for 12 hours or more. In any embodiment disclosed herein, the wound inserts may be administered one, two, three, four, or five times per day. In any embodiment disclosed herein, the wound inserts may be administered daily for one, two, three, four, or five weeks. In any embodiment disclosed herein, the wound inserts may be administered daily for less than 6 weeks. In any embodiment disclosed herein, the wound inserts may be administered daily for 6 weeks or more. In any embodiment disclosed herein, the wound inserts may be administered daily for 12 weeks or more. In any embodiment disclosed herein, the wound inserts may be administered every day, every other day, every third day, every fourth day, every fifth day, or every sixth day. In any embodiment disclosed herein, the wound inserts may be administered weekly, bi-weekly, tri-weekly, or monthly. In any embodiment disclosed herein, the wound inserts may be administered for a chronic wound as appropriate.

In any embodiment herein, the method may include employing the wound insert in the context of a negative-pressure therapy, where the negative-pressure therapy may include positioning the wound insert in and/or proximate to the wound. The negative-pressure therapy may further include sealing the wound insert to tissue surrounding the wound to form a sealed space. For example, the wound insert may be positioned in and/or proximate to the wound and sealed to an attachment surface near the wound, for example, to undamaged epidermis peripheral to a wound.

The negative-pressure therapy method in any embodiment herein may further include fluidly coupling a negative-pressure source to the sealed space and operating the negative-pressure source to generate a negative pressure in the sealed space. For example, the negative-pressure source may be coupled to the wound insert such that the negative-pressure source may be used to reduce the pressure in the sealed space. For example, negative pressure applied across the wound, for example, via the wound insert may be effective to induce macrostrain and microstrain at the wound site, as well as remove exudates and other fluids from the wound.

### Methods of Manufacturing a Wound Insert of the Present Technology

In a further related aspect, a method of manufacturing a wound insert of any embodiment of the present technology is provided, where the method includes: fabricating an inner core of the wound insert from a slurry that includes one or more biopolymers, where in the inner core is in the form of a rope or rod; drying the inner core; fabricating the outer layer that includes a reabsorbable polymer, wherein the outer layer is shaped around the inner core; wherein: the wound insert is an a shape comprising a cylinder, a sphere, a cube, a cuboid, a hexagonal prism, a cone, a square-based pyramid, triangular-based pyramid, or a triangular prism; the outer layer has a modulus of elasticity from about 0.5 MPa to about 5.0 MPa; and the inner core exhibits a lower modulus of elasticity than the outer layer.

In any embodiment herein, the fabricating may include extrusion of the slurry to fabricate the inner core. For example, in any embodiment herein, the slurry may be extruded via die such that the inner core such that the inner core may be the form of a rope or a rod. As disclosed herein, rope or rod form of the inner core may have a shape as disclosed herein in any embodiment, for example, the shape may be a cylinder, a sphere, a cube, a cuboid, a hexagonal prism, a cone, a square-based pyramid, triangular-based pyramid, or a triangular prism.

In any embodiment herein, the slurry may have a pH from about 3 to 7. For example, in any embodiment herein, the slurry may have a pH of about 3, about 3.5, about 4, about 4.5, about 5, about 5.5, about 6, about 6.5, about 7, or any range including and/or in between any two or more of the preceding values. In any embodiment herein, the pH of the slurry may be from about 4.5 to about 5.5.

In any embodiment herein, the slurry corresponds to the resultant inner core. In any embodiment disclosed herein, the slurry may include one or more biopolymers including, but not limited to, a collagen, an oxidized cellulose, an oxidized regenerated cellulose (ORC), a polysaccharide, a chitosan, hyaluronic acid, or a combination of any two or more thereof. In any embodiment disclosed herein, the biopolymer may be collagen. In any embodiment disclosed herein, the biopolymer may include collagen, ORC, or a combination thereof. In any embodiment disclosed herein, the collagen of the slurry may include a mammalian collagen, such as a bovine collagen, a human collagen, or a combination thereof as described herein in any embodiment.

The amount of biopolymer included in the slurry of any embodiment herein may be about 0.01 wt. % to 100 wt. % as described herein in any embodiment. Thus, the amount of collagen may be about 0.01 wt. %, about 0.1 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. % to about 100 wt. % of the biopolymer; thus, the inner core may include the biopolymer at an amount (based on the weight of the inner core) of about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 95 wt. %, about 99 wt. %, about 100 wt. %, or any range including and/or in between any two of these values based on dry weight of the slurry.

The collagen included in the slurry of any embodiment herein may have a weight-average molecular weight of about 5,000, about 6,000, about 7,000, about 8,000, about 9,000, about 10,000, about 12,000, about 14,000, about 16,000, about 18,000, about 20,000, about 22,000, about 24,000, about 26,000, about 28,000, about 30,000, about 32,000, about 34,000, about 36,000, about 38,000, about 40,000, about 45,000, about 50,000, about 55,000, about 60,000, about 65,000, about 70,000, about 75,000, about 80,000, about 85,000, about 90,000, about 95,000, about 100,000, or any range including and/or in between any two of these values.

As discussed above, the slurry may include ORC as described herein in any embodiment. The slurry of any embodiment herein may include about 30 wt. % to about 70 wt. % ORC with a weight-average molecular weight of about 50,000 to about 1,000,000. Thus, the ORC of any embodiment disclosed herein may be included in an amount of about 30 wt. %, about 32 wt. %, about 34 wt. %, about 36 wt. %, about 38 wt. %, about 40 wt. %, about 42 wt. %, about 44 wt. %, about 46 wt. %, about 48 wt. %, about 50 wt. %, about 52 wt. %, about 54 wt. %, about 56 wt. %, about 58 wt. %, about 60 wt. %, about 62 wt. %, about 64 wt. %, about 66 wt. %, about 68 wt. %, about 70 wt. %, or any range including and/or in between any two of these values. The ORC may have a weight-average molecular weight of about 50,000, about 100,000, about 150,000, about 200,000, about 250,000, about 300,000, about 350,000, about 400,000, about 450,000, about 500,000, about 550,000, about 600,000, about 650,000, about 700,000, about 750,000, about 800,000, about 850,000, about 900,000, about 950,000, about 1,000,000, or any range including and/or in between any two of these values.

In any embodiment herein including collagen along with ORC in the slurry, a weight ratio of collagen to ORC may be about 60:40 to about 40:60 in the first slurry and/or the second slurry. The weight ratio of the one or more of collagen and chitosan to ORC may be about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, or any range including and/or in between any two of these values.

In any embodiment disclosed herein, the slurry may further include one or more bioabsorbable polymers, plasticizers, cross-linking agents, functional agents, or combinations of two or more thereof.

In any embodiment disclosed herein, the slurry may further include about 1 wt. % to about 20 wt. % of bioabsorbable polymers as disclosed herein in any embodiment based on dry weight of the slurry. Thus, the total amount of the cross-linking agents may include in the inner core may be about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, or any range including and/or in between any two of these values.

In any embodiment disclosed herein, the slurry may include about 0.01 wt. % to about 10 wt. % of cross-linking agents based on the weight of the inner core. Exemplary cross-linking agents may include, but are not limited to, epichlorhydrin, carbodiimide, hexamethylene diisocyanate (HMDI) orglutaraldehyde, or a combination of any two or more thereof. Thus, the total amount of the cross-linking agents may include in the inner core may be about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, or any range including and/or in between any two of these values.

In any embodiment disclosed herein, the slurry may include about 0.001 wt. % to about 5 wt. % of functional agents. Thus, the total amount of functional agents in the slurry may be about 0.001 wt. %, about 0.002 wt. %, about 0.003 wt. %, about 0.004 wt. %, about 0.005 wt. %, about 0.006 wt. %, about 0.007 wt. %, about 0.008 wt. %, about 0.009 wt. %, about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, or any range including and/or in between any two of these values.

Following fabrication of the inner core, the method includes drying the inner core. For example, in any embodiment herein, drying the inner core may include freeze-drying the inner core. The drying may be carried out by heating the inner core at a temperature from about 20° C. to about 80° C. Suitable drying temperatures in any embodiment herein may include, but are not limited to, about 20° C., about 25° C., about 30° C., about 35° C., about 40° C., about 45° C., about 50° C., about 55° C., about 60° C., about 65° C., about 70° C., about 75° C., about 80° C. or any range including and/or in between any two of the preceding values. The drying may be carried out for a duration of about 2 hours to about 24 hours. For example, in any embodiment herein, the drying may be carried out for about 2 hours, about 3 hours, about 4 hour, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or any range including and/or in between any two of the preceding values.

In any embodiment disclosed herein, fabricating the outer layer may include extruding, braiding, laminating, or coating the bioabsorbable polymer around the inner core. For example, in any embodiment herein, the fabricating may include co-extruding the inner core with the bioabsorbable polymer, where the bioabsorbable polymer forms a coating around the inner core. In any embodiment disclosed herein, fabricating the outer layer may include braiding fibers of the bioabsorbable polymer around the inner core. In any embodiment disclosed herein, the fabricating may include forming a hollow sheath of the bioabsorbable material having a hollow center. For example, in any embodiment disclosed herein, the hollow sheath may extruded using a die to provide the hollow shape or via blown film extrusion. The outer layer may be shaped around the inner core by inserting the inner core into the hollow center of the outer layer sheath. In any embodiment disclosed herein, fabricating the outer layer may include lamination (e.g., hot lamination) of one or more sheets of the bioabsorbable polymer to form the outer layer. For example, in any embodiment disclosed herein, the bioabsorbable polymer of the outer layer may be in the form of a solid or mesh sheet, which may be laminated to form the outer layer around the inner core of the wound insert. Laminating the sheets of bioabsorbable polymer to form the outer layer may include forming a weld or joint that includes excess outer layer (see FIGURE).

In any embodiment disclosed herein, fabricating the outer layer may include coating the bioabsorbable polymer onto the outer facing surface of the inner core. For example, in any embodiment herein, the bioabsorbable polymer may be placed in solution and coated onto the inner surface via coating techniques known in the art (e.g., curtain or spray techniques).

In a related aspect, the present disclosure provides a wound insert prepared according to the method of manufacturing said wound insert as described herein in any embodiment.

### Kits Comprising the Wound Insert of the Present Technology

In a further related aspect, the present disclosure provides kits that include a wound insert of any embodiment disclosed herein and instructions for use. The kits of the present technology may also include methods for treating a wound in a subject in need thereof. The kit may optionally comprise components such as antiseptic wipes, ointment, adhesive tape, tweezers, scissors, etc.

### EQUIVALENTS

While certain embodiments have been illustrated and described, a person with ordinary skill in the art, after reading the foregoing specification, can effect changes, substitutions of equivalents and other types of alterations to the compositions, systems, and methods of the present technology. Each aspect and embodiment described above can also have included or incorporated therewith such variations or aspects as disclosed in regard to any or all of the other aspects and embodiments.

The present technology is also not to be limited in terms of the particular aspects and embodiments described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety to the extent they are not inconsistent with the explicit teachings of this specification. Definitions that are contained in text incorporated by reference are excluded to the extent that they contradict definitions in this disclosure.

Other embodiments are set forth in the following claims, along with the full scope of equivalents to which such claims are entitled.

What is claimed is:

1. A wound insert comprising:
an outer layer comprising one or more bioabsorbable polymers, wherein the outer layer has a modulus of elasticity of about 0.5 MPa to about 5.0 MPa; and
an inner core comprising a biopolymer, wherein the inner core has a lower modulus of elasticity than the outer layer.

2. The wound insert of claim 1, wherein the outer layer has a modulus of elasticity of about 1.0 MPa to about 2.5 MPa.

3. The wound insert of claim 1 or claim 2, wherein the wound insert is in a shape of a cylinder, a cylinder with at least one rounded edge, a cuboid, or a cuboid with at least one rounded edge.

4. The wound insert of any one of claims 1-3, wherein the wound insert is in a shape comprising a cuboid.

5. The wound insert of any one of claims 1-4, wherein the wound insert has a width and/or height of about 0.1 cm to about 3 cm.

6. The wound insert of any one of claims 1-5, wherein the width and/or height of the wound insert is about 0.3 cm to about 1.5 cm.

7. The wound insert of any one of claims 1-6, wherein the width and/or height of the wound insert is about 0.5 cm to about 1 cm.

8. The wound insert of any one of claims 1-7, wherein the wound insert has a length of about 1 cm to about 10 cm.

9. The wound insert of any one of claims 1-8, wherein the length of the wound insert is about 2 cm to about 5 cm.

10. The wound insert of any one of claims 1-9, wherein the outer layer has a thickness of about 20 μm to about 1.5 mm.

11. The wound insert of any one of claims 1-10, wherein the thickness of the outer layer is about 50 μm to about 1 mm.

12. The wound insert of any one of claims 1-11, wherein the thickness of the outer layer is about 100 μm to about 300 μm.

13. The wound insert of any one of claims 1-12, wherein at least the outer layer of the wound insert comprises perforations.

14. The wound insert of any one of claims 1-13, wherein the wound insert comprises through-holes through the entire insert.

15. The wound insert of any one of claims 1-14, wherein the through-holes have a width and/or height of about 2 mm to about 8 mm.

16. The wound insert of any one of claims 1-15, wherein the one or more bioabsorbable polymers of the outer layer comprise polylactic acid (PLA), gelatin, polyglycolide, polycaprolactone, or a combination of any two or more thereof.

17. The wound insert of any one of claims 1-16, wherein the bioabsorbable polymer of the outer layer comprises PLA, gelatin, or a combination thereof.

18. The wound insert of any one of claims 1-17, wherein the outer layer comprises about 0.01 wt. % to about 100 wt. % of the one or more bioabsorbable polymers by weight of the outer layer.

19. The wound insert of any one of claims 1-18, wherein the outer layer comprises PLA.

20. The wound insert of any one of claims 1-19, wherein the outer layer comprises gelatin.

21. The wound insert of any one of claims 1-20, wherein the outer layer further comprises one or more of collagen, plasticizers, functional agents, or a combination of two or more thereof.

22. The wound insert of claim 1-21, wherein the outer layer comprises about 0.001 wt. % to about 10 wt. % of collagen based on the weight of the outer layer.

23. The wound insert of claim 21 or claim 22, wherein the one or more functional agents comprises antimicrobial agents, growth factors, peptides, antioxidants, or combinations of any two or more thereof.

24. The wound insert of any one of claims 1-23, wherein the outer layer comprises about 0.001 wt. % to about 5 wt. % of one or more functional agents based on the weight of the outer layer.

25. The wound insert of any one of claims 1-24, wherein the outer layer comprises about 1 wt. % to about 20 wt. % of plasticizer based on the weight of the outer layer.

26. The wound insert of claim 25, wherein the plasticizer comprises glycerin, polyethylene oxide, or a combination thereof.

27. The wound insert of any one of claims 1-26, wherein the biopolymer of the inner core comprises collagen, an oxidized cellulose, an oxidized regenerated cellulose (ORC), a polysaccharide, a chitosan, hyaluronic acid, or a combination of any two or more thereof.

28. The wound insert of any one of claims 1-27, wherein the inner core comprises about 0.01 wt. % to about 100 wt. % of the biopolymer based on the weight of the inner core.

29. The wound insert of any one of claims 1-28, wherein the inner core comprises about 10 wt. % to about 50 wt. % of the biopolymer based on the weight of the inner core.

30. The wound insert of any one of claims 1-29, wherein the inner core comprises collagen.

31. The wound insert of any one of claims 1-30, wherein the inner core comprises collagen and ORC.

32. The wound insert of any one of claims 1-31, wherein the inner core comprises collagen and ORC in a weight ratio of collagen to ORC of about 60:40 to about 40:60.

33. The wound insert of any one of claims 1-32, wherein the inner core further comprises one or more of bioabsorbable polymers, cross-linking agents, plasticizers, functional agents, or combinations of any two or more thereof.

34. The wound insert of any one of claims 1-33, wherein the inner core comprises about 1 wt. % to about 20 wt. % of bioresorbable polymer based on the weight of the inner core.

35. The wound insert of any one of claims 1-34, wherein the inner core comprises about 0.001 wt. % to about 5 wt. % of one or more functional agents based on the weight of the inner core.

36. The wound insert of any one of claims 1-35, wherein the inner core comprises about 1 wt. % to about 20 wt. % of plasticizer based on the weight of the inner core.

37. The wound insert of any one of claims 1-36, wherein the inner core is the form of a rope or rod, wherein the rope and/or rod conforms to the overall shape of the wound insert.

38. A method for treating a wound in a subject in need thereof, the method comprising administering to the wound a wound insert of any one of claims 1-37.

39. The method of claim 38, wherein the wound is a tunneling wound, a dermal wound, a diabetic wound, an acute wound, a chronic wound, or a combination of any two or more thereof.

40. The method of claim 38 or claim 39, wherein the wound is a tunneling wound.

41. The method of any one of claims 38-40, wherein the wound insert is administered directly to the wound.

42. The method of any one of claims 38-41, further comprising administering negative pressure to the wound subsequent to administering the wound insert.

43. A method of manufacturing a wound insert comprising:
fabricating an inner core of the wound insert from a slurry comprising one or more biopolymers, wherein the inner core is in the form of a rope or rod;
drying the inner core;
fabricating the outer layer comprising a reabsorbable polymer, wherein the outer layer is shaped around the inner core;
wherein:
the wound insert is an a shape comprising a cylinder, a sphere, a cube, a cuboid, a hexagonal prism, a cone, a square-based pyramid, triangular-based pyramid, or a triangular prism;
the outer layer has a modulus of elasticity from about 0.5 MPa to about 5.0 MPa; and
the inner core exhibits a lower modulus of elasticity than the outer layer.

44. The method of claim 43, wherein the slurry has a pH from about 3 to 7.

45. The method of claim 43 or claim 44, wherein the slurry comprises one or more biopolymers comprising collagen, oxidized cellulose, oxidized regenerated cellulose (ORC), polysaccharide, chitosan, hyaluronic acid, or a combination of any two or more thereof.

46. The method of any one of claims 43-45, wherein the slurry comprises about 0.01 wt. % to about 100 wt. % of the biopolymer based on dry weight of the slurry.

47. The method of any one of claims 43-46, wherein the slurry further comprises one or more bioabsorbable polymers, plasticizers, cross-linking agents, functional agents, or combinations of two or more thereof.

48. The method of any one of claims 43-47, wherein the slurry comprises about 1 wt. % to about 20 wt. % of bioabsorbable polymers based on dry weight of the slurry.

49. The method of any one of claims 43-48, wherein the slurry comprises about 0.01 wt. % to about 10 wt. % of cross-linking agent based on dry weight of the slurry.

50. The method of any one of claims 43-49, wherein the slurry comprises about 0.001 wt. % to about 5 wt. % of functional agents based on dry weight of the slurry.

51. The method of any one of claims 43-50, wherein the slurry comprises collagen and ORC, wherein a weight ratio of collagen to ORC is about 60:40 to about 40:60.

52. The method of any one of claims 43-51, wherein the slurry comprises about 10 wt. % to about 50 wt. % of the biopolymer based on dry weight of the slurry.

53. The method of any one of claims 43-52, wherein the drying the inner core comprises heating or freeze drying the inner core.

54. The method of any one of claims 43-53, wherein fabricating the outer layer comprises extruding, braiding, laminating, or coating the bioabsorbable polymer around the inner core.

55. The method of any one of claims 43-54, wherein fabricating the outer layer comprises forming a sheath having a hollow center, wherein the inner core is inserted into the hollow center of the sheath.

56. A wound insert prepared according to the method of any one of claims 43-54.

57. A kit comprising
a wound insert of any one of claims 1-37; and
instructions for use.

58. The kit of claim 57, wherein the instructions for use comprise instructions for a method according to any one of claims 38-42.

59. A kit comprising
a wound insert of claim 56; and
instructions for use.

60. The kit of claim 59, wherein the instructions for use comprise instructions for a method according to any one of claims 38-42.
